(19) United States
(12) Patent Application Publication
(10) Pub. No.: US 2025/0339858 A1
(43) Pub. Date: Nov. 6, 2025

(54) System and method of tracking and checking specimen

(71) Applicant: JAG Technology Ltd., New Taipei City (TW)

(72) Inventor: Tien Hsiu TSO, New Taipei City (TW)

(21) Appl. No.: 18/826,450

(22) Filed: Sep. 6, 2024

(30) Foreign Application Priority Data
May 3, 2024 (TW) 113116476

(51) Int. Cl. B01L 3/00

(52) U.S. Cl. CPC B01L 3/508; B01L 3/505; B01L 2200/185; B01L 2300/021

(57) ABSTRACT

A system and method of tracking and checking specimen are disclosed. It is suitable for at least a specimen container, which is transported from a transmitting end to a checking end. The system comprises a weight-sensing module, an image capturing module, a controlling module, and a network communication module, which are disposed at the transmitting end and the checking end. In accordance with a weight of the specimen container and an identified target image, the controlling module generates first specimen information and second specimen information respectively at the transmitting end and the checking end. The checking end is further equipped with a comparison module for comparing whether the first specimen information and the second specimen information are identical.

### TECHNICAL FIELD

The application relates generally to an automatic tracking and checking system and method, and more particularly, to a system and method of tracking and checking specimen.

### BACKGROUND OF THE INVENTION

In a conventional testing process, after a specimen container equipped with specimen received by medical personnel, the specimen container equipped with specimen is usually transported to an inspection unit by manual transportation for inspection by the inspection personnel. The process mainly includes scanning barcode on the specimen container, and identifying quantity and integrity of packages through manual operation.

However, for hospitals or other medical units with a shortage of manpower, it is very difficult to spend manpower for collecting and transporting specimen containers containing specimens. Therefore, a certain amount of waste may accumulate at the examination counter. After a certain number of specimen containers loaded with specimens are collected, the accumulated specimen containers loaded with specimens are then transported to the inspection unit by manpower at one time. However, such an approach may cause incorrectly taking/sending or stealthily substitute due to manual operation. Therefore, it will cause unexpected situation.

### SUMMARY OF THE INVENTION

To address the deficiencies of conventional problem, an embodiment of the invention provides a system of tracking and checking specimen, which is suitable to at least one specimen container transported from a transmitting end to a checking end, the system comprises a weight sensing module disposed at the transmitting end and the checking end, to measure a weight of the at least one specimen container. The system comprises: a weight sensing module disposed at the transmitting end and the checking end, to measure a weight of the at least one specimen container; an image capturing module disposed above the weight sensing module, to capture a target image of the at least one specimen container; an image identification module disposed at the transmitting end and the checking end, the image identification module is electrically connected to the image capturing module for identifying the target image; a control module disposed at the transmitting end and the checking end, the control module is electrically connected to the weight sensing module and the image identification module for generating a first specimen information and a second specimen information respectively based on the weight and an identified target image of the at least one specimen container; and a network communication module disposed at the transmitting end and the checking end, to transmit the first specimen information from the transmitting end to the checking end through a network for receiving the first specimen information, wherein the system further comprising a comparison module disposed at the checking end, to compare whether the first specimen information matches the second specimen information.

In another aspect of the application, the system further comprises a warning module, the warning module issues warning messages when the first specimen information does not match the second specimen information.

In another aspect of the application, the system further comprises at least one storage module to store the first specimen information and the second specimen information.

In another aspect of the application, the system further comprises at least one display module electrically connected to the control module, to display the first specimen information and the second specimen information.

In another aspect of the application, the system further comprises at least one input module electrically connected to the control module, to input operation time or user identity.

In another aspect of the application, the image capturing module is an electronic scale.

In another aspect of the application, the at least one specimen container is one of tube, specimen box, specimen can, urine cup and specimen bag.

In another aspect of the application, the first specimen information and the second specimen information comprise a weight and an appearance image of the at least one specimen container.

In another aspect of the application, the image capturing module comprises a photographing lens for capturing images.

As stated above, a system and method provided by this invention s can achieve improved efficiency of collecting and transporting specimen. In its overall implementation, practical functional characteristics can be improved.

Other aspects and features of the application will become apparent to those with ordinary skill in the art upon review of the following descriptions of specific embodiments of the specimen tracking and checking system without departing from the scope and spirit as set forth.

### BRIEF DESCRIPTION OF THE DRAWINGS

FIG. 1 is an architecture diagram of a system of tracking and checking specimen according to an embodiment of the invention;

FIG. 2 is a perspective view schematically showing an image capturing module and a weight sensing module of the specimen tracking and checking system according to an embodiment of the invention;

FIG. 3 is a perspective view schematically showing image capturing module and weight sensing module of specimen tracking and checking system according to another embodiment of the invention;

FIG. 4 is a perspective view schematically showing image capturing module and weight sensing module of specimen tracking and checking system according to another embodiment of the invention.

### DETAILED DESCRIPTION

The making and using of the embodiments of the system and method of tracking and checking specimen is discussed in detail below. It should be appreciated, however, that the embodiments provide many applicable inventive concepts that can be embodied in a wide variety of specific contexts. The specific embodiments discussed are merely illustrative of specific ways to make and use the embodiments, and do not limit the scope of the disclosure.

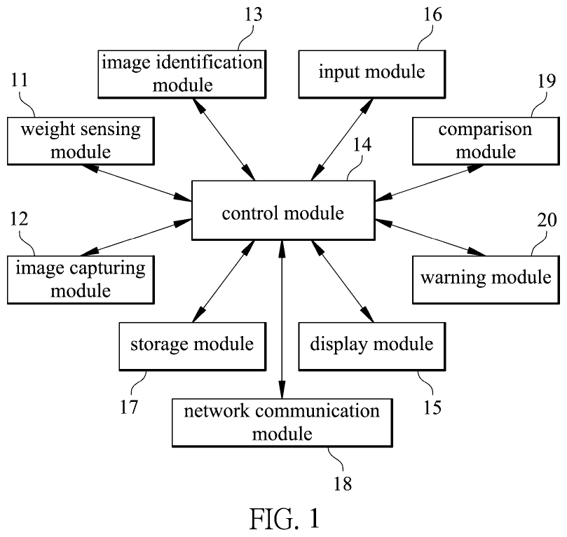

Referring to FIG. 1, FIG. 1 is an architecture diagram of a system of tracking and checking specimen according to an embodiment of the invention. It is suitable for at least a specimen container transported from a transmitting end to a checking end. For example, the transmitting end is an operation room for executing specimen collection. For example, the checking end is an intermediate station (as an intermediary storage place) or a laboratory. The specimen container is capable of collecting specimen such as blood, urine or phlegm. The system comprises a weight sensing module 11, an image capturing module 12, an image identification module 13, a control module 14, a display module 15, an input module 16, a storage module 17, a network communication module 18, a comparison module 19, and a warning module 20. The control module 14 is respectively electrically connected to the weight sensing module 11, the image capturing module 12, the image identification module 13, the display module 15, the input module 16, the storage module 17, the network communication module 18, the comparison module 19 and the warning module 20 for controlling procedure among each module.

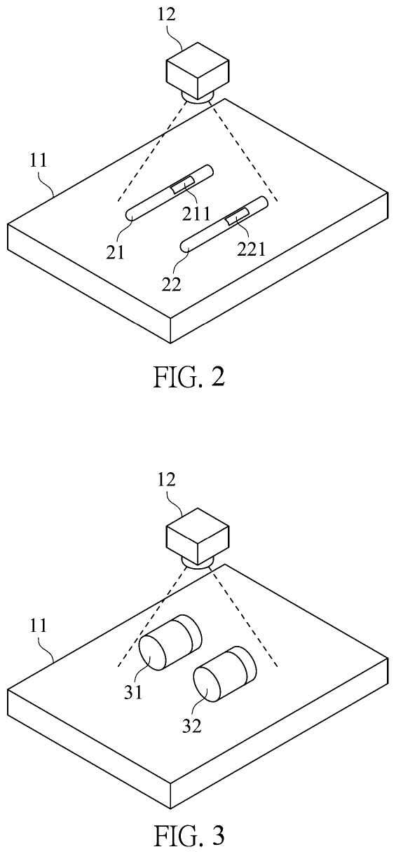

Referring to FIG. 2, FIG. 2 is a perspective view schematically showing image capturing module and weight sensing module of system of tracking and checking specimen according to an embodiment of the invention. At the transmitting end, the weight sensing module 11 executes weight measurement of specimen container 21 and specimen container 22 for obtaining original weight. The image capturing module 12 captures a target image (for example, a picture recording original images of appearance) of specimen container 21 and specimen container 22. The image capturing module 12 is disposed above the weight sensing module 11. The specimen container 201 and 202 are tubes. For example, the weight sensing module 11 is an electronic scale. For example, the image capturing module 12 is a camera lens. Specimen container 21 and specimen container 22 can be attached with label 211 and label 221, respectively. Label 211 and label 221 may be one of two-dimensional barcode, Quick Response (QR) code, Radio Frequency Identification (RFID) label or Near Field Communication (NFC) label. Label 211 and label 221 include information, such as specimen number, description of the specimen, personnel identity (such as sampling personnel/packer), location (such as operation room/), and time (such as sampling time).

Please refer to FIG. 1 and FIG. 2. The weight sensing module 11, the image capturing module 12, the image identification module 13, the control module 14, the display module 15, the input module 16, the storage module 17 and the comparison module 18 are disposed at the transmitting end. In accordance with the target image captured by the image capturing module 12, image identification module 13, which is electrically connected to the image capturing module 12, obtains an identified target image including appearance information such as tube length, shape and color of specimen container 201 and specimen container 202.

In accordance with the measured weight and the identified target image of specimen container 201 and specimen container 202, the control module 14 generates first specimen information. The first specimen information includes at least appearance and weight of the specimen container at the transmitting end. The display module 15 is electrically connected to the control module 14 for displaying the first specimen information. The input module 16 is electrically connected to the control module 14 so that users can input information such as user identity and operating time on an operation interface of input module 16. The storage module 17 is electrically connected to the control module 14 for storing information such as the first specimen information. The network communication module 18 sends the first specimen information to a checking end or a remote sever platform (not shown) through network. Accordingly, many people can immediately search or track information of multiple specimens at different places.

It should be understood that the components described in the embodiment of FIG. 1 are for illustrative purposes only and are not intended to limit the scope of the application. For example, the control module 14 may include various circuit logic for transmitting control signal to the image capturing module 12, receiving image data from the image capturing module 12, controlling the network communication module 18 to obtain network connection, and read or store data from the storage module 17. The storage module 17 may be non-transitory computer-readable storage medium including: memory (such as flash memory, Non-volatile Random Access Memory (NVRAM)) or magnetic storage apparatus (such as drive, disk) or combination of the above medium for storing image data.

In a preferred embodiment, the network communication module 18 may support cellular communication network such as Global System for Mobile communication (GSM), Long Term Evolution (LTE) or LTE-Advanced (LTE-A), or transmission communication such as wireless LAN (also known as Wi-Fi) or Bluetooth.

In addition to the weight sensing module, the image capturing module, the image identification module, the control module, the display module, the input module, the storage module, the network communication module, a comparison module 19 and warning module 20 are further disposed at the checking end. After the specimen container 21 and specimen container 22 transported from the transmitting end to the checking end (for example, by specimen transfer box), labels (not shown) are attached on the specimen container 21 and specimen container 22 respectively. The labels include information, such as specimen number, description of the specimen, personnel identity (such as handler/receiver), location (such as relay storage location/Pathology Department), and time (such as arrival time/departure time/receiving time). At the checking end, second specimen information is generated through the same procedure as the transmitting end. The second specimen information includes at least weight and appearance of the specimen container at the checking end. The comparison module 19 compares the second specimen information with the first specimen information received from the transmitting end. When comparison result of the first specimen information and the second specimen information is not consistent, the warning module 20 issues a warning message for reminding weight/appearance of specimen container 21 and specimen container 22 is different from original weight/appearance. It indicates quantity/content of specimen container 21 and specimen container 22 is incorrect. The warning message can be shown on the display module of the checking end or sent to the remote sever platform.

Please refer to FIG. 3. FIG. 3 is a perspective view schematically showing image capturing module and weight sensing module of specimen tracking and checking system according to an embodiment of the invention. In this embodiment, specimen container 31 and specimen container 32 is urine cup. In accordance with the target image captured by the image capturing module 12, image identification module 13 obtains an identified target image including appearance information such as cup length, shape and color of specimen container 31 and specimen container 32.

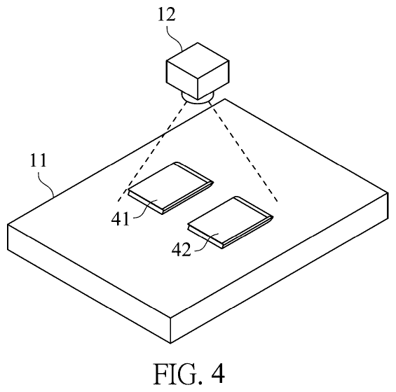

Referring to FIG. 4, FIG. 4 is a perspective view schematically showing image capturing module and weight sensing module of specimen tracking and checking system according to another embodiment of the invention. In this embodiment, specimen container 41 and specimen container 42 are specimen bags. In accordance with the target image captured by the image capturing module 12, image identification module 13 obtains an identified target image including appearance information such as size and color of specimen container 41 and specimen container 42.

The application also provides a specimen tracking and checking method. An embodiment of the invention provides a specimen tracking and checking method, which is suitable to at least one specimen container transported from a transmitting end to a checking end, the method comprising: measuring a weight of the at least one specimen container by a weight sensing module disposed at the transmitting end, capturing a target image of the at least one specimen container by an image capturing module disposed at the transmitting end, identifying the target image by an image identification module disposed at the transmitting end, generating a first specimen information by a control module disposed at the transmitting end based on the weight and an identified target image of the at least one specimen container, transmitting the first specimen information to the checking end by a network communication module disposed at the transmitting end, measuring a weight of the at least one specimen container by a weight sensing module disposed at the checking end, capturing a target image of the at least one specimen container by an image identification module disposed at the checking end, identifying the target image by an image identification module disposed at the checking end, generating a second specimen information by a control module disposed at the checking end based on a weight and an identified target image of the at least one specimen container at the checking end; and comparing whether the first specimen information matches the second specimen information by a comparison module disposed at the checking end.

It should be understood that specimen tracking and checking system of this application can determine whether quantity of specimen containers is correct by comparing consistency of weight and appearance image of specimen container between the transmitting end and the checking end. In accordance with system and method of pathology sample digital track and automatic inspection, users can precisely record and check weight and appearance of specimen container at various places. The use of specimen tracking and checking system can not only effectively reduce labor costs, but also significantly reduce the occurrence of errors such as changeling, lost and misplace. Precision and safety of specimen transferring process can be significantly reduced.

While the application has been described by way of example and in terms of preferred embodiment, it should be understood that the application cannot be limited thereto. Those who are skilled in this technology can still make various alterations and modifications without departing from the scope and spirit of this application. Therefore, the scope of the present application shall be defined and protected by the following claims and their equivalents.

What is claimed is:

1. A system of tracking and checking specimen, which is suitable to at least one specimen container transported from a transmitting end to a checking end, the system comprising:
a weight sensing module disposed at the transmitting end and the checking end, to measure a weight of the at least one specimen container;
an image capturing module disposed above the weight sensing module, to capture a target image of the at least one specimen container;
an image identification module disposed at the transmitting end and the checking end, the image identification module is electrically connected to the image capturing module for identifying the target image;
a control module disposed at the transmitting end and the checking end, the control module is electrically connected to the weight sensing module and the image identification module for generating a first specimen information and a second specimen information respectively based on the weight and an identified target image of the at least one specimen container; and
a network communication module disposed at the transmitting end and the checking end, to transmit the first specimen information from the transmitting end to the checking end through a network for receiving the first specimen information,
wherein the system further comprising a comparison module disposed at the checking end, to compare whether the first specimen information matches the second specimen information.

2. The system of claim 1, further comprising a warning module, the warning module issues warning messages when the first specimen information does not match the second specimen information.

3. The system of claim 1, further comprising at least one storage module to store the first specimen information and the second specimen information.

4. The system of claim 1, further comprising at least one display module electrically connected to the control module, to display the first specimen information and the second specimen information.

5. The system of claim 1, further comprising at least one input module electrically connected to the control module, to input operation time or user identity.

6. The system of claim 1, wherein the image capturing module is an electronic scale.

7. The system of claim 1, wherein the at least one specimen container is one of tube, specimen box, specimen can, urine cup and specimen bag.

8. The system of claim 1, wherein the first specimen information and the second specimen information comprise a weight and an appearance image of the at least one specimen container.

9. The system of claim 1, wherein the image capturing module comprises a photographing lens for capturing images.

10. A method of tracking and checking specimen, which is suitable to at least one specimen container transported from a transmitting end to a checking end, the method comprising:
measuring a weight of the at least one specimen container by a weight sensing module disposed at the transmitting end;
capturing a target image of the at least one specimen container by an image capturing module disposed at the transmitting end;
identifying the target image by an image identification module disposed at the transmitting end;
generating a first specimen information by a control module disposed at the transmitting end based on the weight and an identified target image of the at least one specimen container;
transmitting the first specimen information to the checking end by a network communication module disposed at the transmitting end;
measuring a weight of the at least one specimen container by a weight sensing module disposed at the checking end;
capturing a target image of the at least one specimen container by an image identification module disposed at the checking end;
identifying the target image by an image identification module disposed at the checking end;
generating a second specimen information by a control module disposed at the checking end based on a weight and an identified target image of the at least one specimen container at the checking end; and
comparing whether the first specimen information matches the second specimen information by a comparison module disposed at the checking end.
